(19) United States
(12) Patent Application Publication
(10) Pub. No.: US 2026/0306289 A1
(43) Pub. Date: Oct. 8, 2026

(54) Muffling System for an Ostomy Pouch

(71) Applicant: Justin Thomas Baker, Erie, PA (US)

(72) Inventor: Justin Thomas Baker, Erie, PA (US)

(21) Appl. No.: 19/572,819

(22) Filed: Mar. 19, 2026

Related U.S. Application Data
(60) Provisional application No. 63/784,746, filed on Apr. 7, 2025.

(51) Int. Cl. A61F 5/44; A61F 5/449

(52) U.S. Cl. CPC A61F 5/4404; A61F 5/449

(57) ABSTRACT

A muffling system for an ostomy pouch is a system designed to be retrofitted to any commonly used ostomy pouches. The system is designed to stifle sounds escaping through the stoma covered by the specific ostomy pouch that patient uses. The system includes a first disc-shaped muffler, a second disc-shaped muffler, and an ostomy pouch. The first disc-shaped muffler and the second disc-shaped muffler correspond to two interfaces designed to apply localized pressure on the stoma through the ostomy pouch without compromising the seal nor the integrity of the ostomy pouch. The ostomy pouch can be any of the ostomy pouches commonly used in ostomy procedures. The ostomy pouch can also be customized for specific use with the first disc-shaped muffler and the second disc-shaped muffler of the system.

The current application claims a priority to the U.S. provisional patent application Ser. No. 63/784,746 filed on Apr. 7, 2025.

### FIELD OF THE INVENTION

The present invention generally relates to wearable medical equipment and muffling devices. More specifically, the present invention provides a wearable muffling system for ostomy pouches designed to mute stoma noises while the ostomy pouch is being worn by the patient.

### BACKGROUND OF THE INVENTION

Ostomy procedures generally involve the creation of a stoma on the patient's body to enable access to the target internal organ through the created stoma. For procedures like a colostomy or an ileostomy, a stoma facilitates the discarding of waste when the patient's body cannot do so normally. An ostomy pouch, also known as stoma bag, is often used with these procedures to collect the waste being discarded through the stoma. Unfortunately, currently available ostomy pouches have many issues and disadvantages. Ostomy patients often experience discomfort due to stoma-related noises caused by the movement of gas, fluids, and solid waste through the stoma. The primary cause of these noises is the involuntary muscle vibrations surrounding the stoma, such as bowel movements. Existing ostomy pouches have failed to prevent these high intensity stoma noises.

Moreover, ostomy pouches often have a bulky design which make ostomy pouches difficult to conceal under normal clothes. Patients often wear loose clothes to conceal the ostomy pouch. Ostomy pouches also often require regular cleaning and care to remain free from odor. Further, prolonged use of current ostomy pouches may lead to skin irritation. Therefore, there is a need for a novel mechanism that muffles stoma noises to make wearing an ostomy pouch more comfortable for the patient.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

The present disclosure provides a muffling system for an ostomy pouch. The muffling system for an ostomy pouch is designed to stifle stoma noises to make wearing an ostomy pouch more comfortable for the patient. In the preferred embodiment, the present invention is designed to be used with traditional ostomy pouches without altering the existing structure of the ostomy pouch. To do so, the present invention comprises a pair of disc-shaped mufflers designed to be attachable to the ostomy pouch. A first disc-shaped muffler is configured for applying gentle pressure against the stoma to control stoma vibrations. The first disc-shaped muffler is externally attached to the ostomy pouch to secure the first disc-shaped muffler to the ostomy pouch. The structure of the first disc-shaped muffler is configured to adapt to a contour of the stoma to provide full coverage to the stoma. Moreover, the second disc-shaped muffler is configured to apply additional external pressure against the stoma to control the stoma vibrations from outside the ostomy pouch.

The second disc-shaped muffler is externally attached to the ostomy pouch and coincides with the first disc-shaped muffler. The second disc-shaped muffler is preferably bigger in diameter than the first disc-shaped muffler, which results in the outer edge of the second disc-shaped muffler protruding past the outer edge of the first disc-shaped muffler. As a result, the second disc-shaped muffler surrounds the first disc-shaped muffler to apply external pressure to the first disc-shaped muffler. Furthermore, a compression device such as a compression band or a compression shirt can be used to press all components together, including the ostomy pouch, to further press the first disc-shaped muffler against the stoma.

Both the foregoing summary and the following detailed description provide examples and are explanatory only. Accordingly, the foregoing summary and the following detailed description should not be restrictive. Further, features or variations may be provided in addition to those set forth herein. For example, embodiments may be directed to various feature combinations and sub-combinations described in the detailed description.

### BRIEF DESCRIPTION OF THE DRAWINGS

FIG. 1 is a top-front-left exploded perspective view of the system of the present invention.

FIG. 2 is a top-rear-right exploded perspective view of the system of the present invention.

FIG. 3 is a top-rear-right perspective view of the system of the present invention, wherein the first disc-shaped muffler and the second disc-shaped muffler are shown attached to the ostomy pouch.

FIG. 4 is a left view of the system of the present invention thereof.

FIG. 5 is a left view of the system of the present invention thereof, wherein the first disc-shaped muffler is shown pressed against the ostomy pouch by the second disc-shaped muffler.

FIG. 6 is a front view of the system of the present invention thereof.

FIG. 7 is a cross-sectional view taken along line 7 -7 in FIG. 6.

FIG. 8 is an illustration showing the system of the present invention, wherein the system is shown worn by a patient, wherein the piece of elastic compression fabric is shown wrapped over the system, and wherein the piece of elastic compression fabric is shown as a compression band.

FIG. 9 is an illustration showing the system of the present invention, wherein the system is shown worn by a patient, wherein the piece of elastic compression fabric is shown wrapped over the system, and wherein the piece of elastic compression fabric is shown as a wearable compression garment.

FIG. 10 is a front view of the second disc-shaped muffler of the system of the present invention.

FIG. 11 is a left view of the second disc-shaped muffler of the system of the present invention.

FIG. 12 is a front view of the first disc-shaped muffler of the system of the present invention.

FIG. 13 is a left view of the first disc-shaped muffler of the system of the present invention.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is it to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

The present disclosure includes many aspects and features. Moreover, while many aspects and features relate to, and are described in the context of the disclosed use cases, embodiments of the present disclosure are not limited to use only in this context.

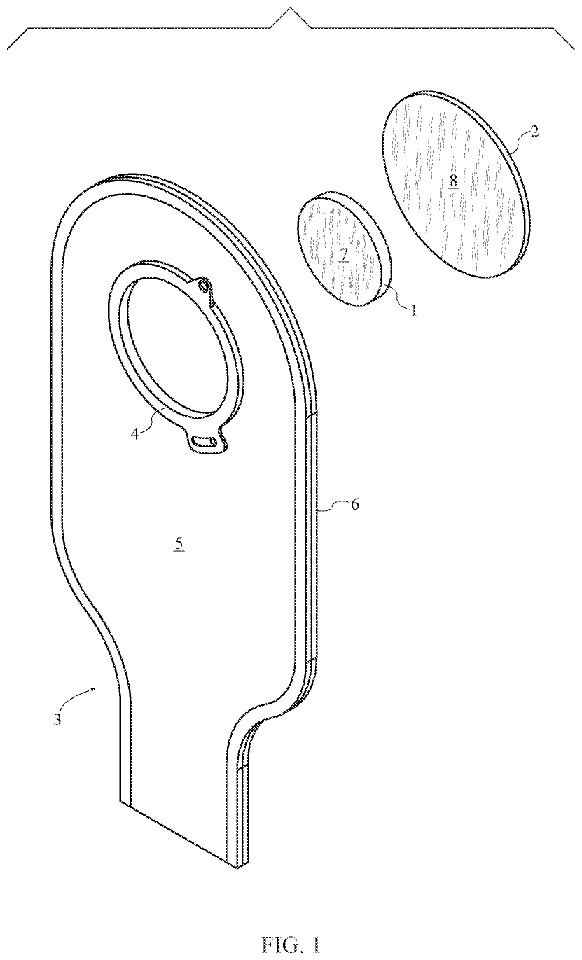

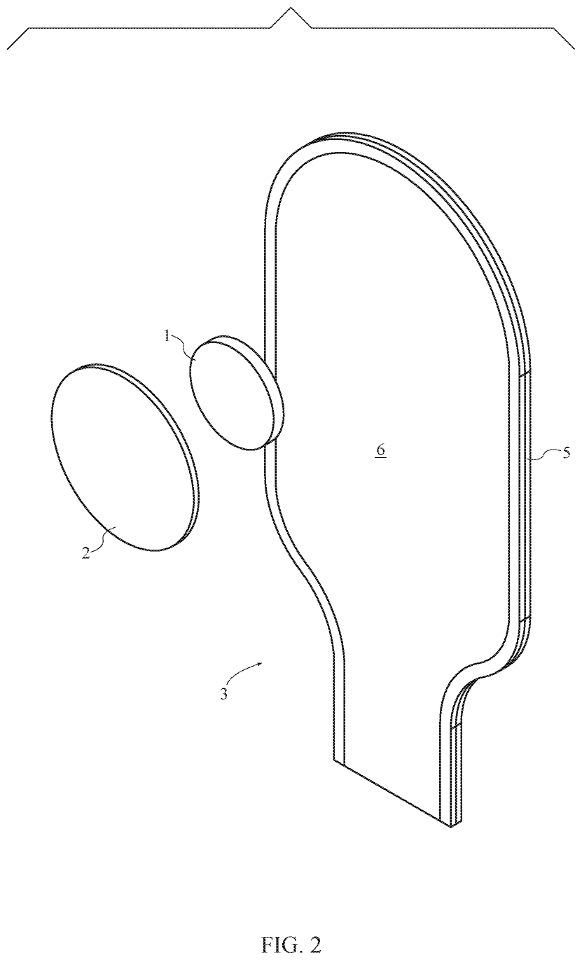

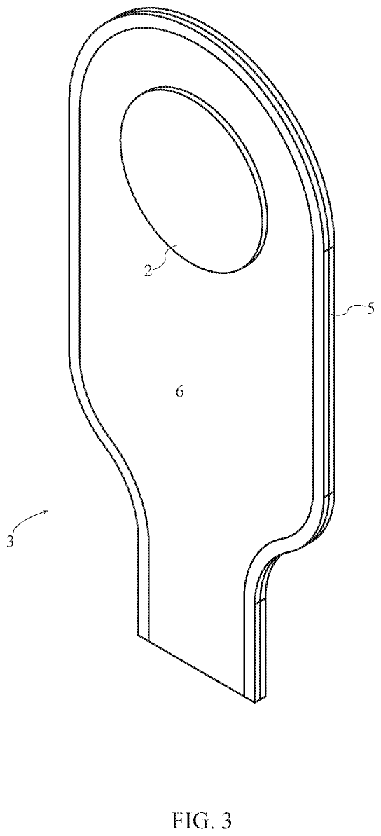

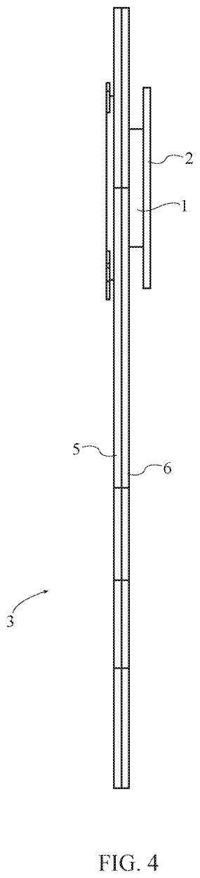

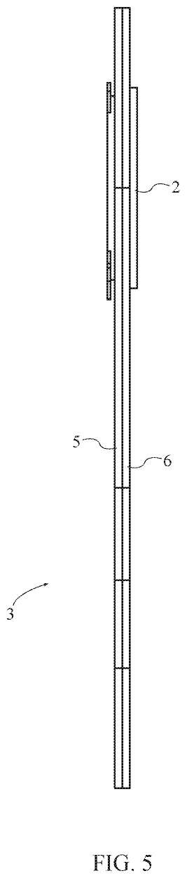

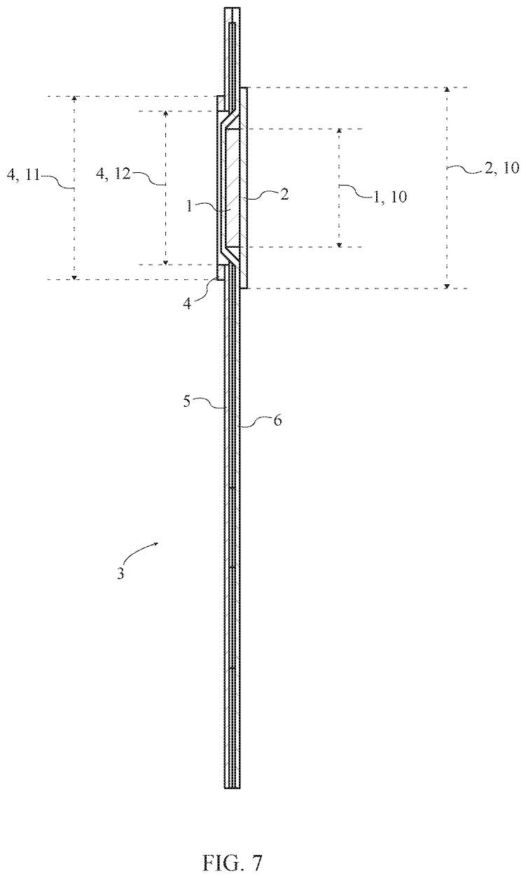

The present invention provides a muffling system for an ostomy pouch. The muffling system is designed to be retrofitted to any commonly used ostomy pouches available on the market. In addition, the muffling system is designed to stifle sounds escaping through the stoma covered by the patient's ostomy pouch. In the preferred embodiment, the present invention comprises a first disc-shaped muffler 1, a second disc-shaped muffler 2, and an ostomy pouch 3, as can be seen in FIGS. 1 through 9. The first disc-shaped muffler 1 and the second disc-shaped muffler 2 correspond to two porous and flexible interfaces designed to apply localized pressure on the stoma through the ostomy pouch 3 without compromising the seal nor the integrity of the ostomy pouch 3. The ostomy pouch 3 can be any of the ostomy pouches commonly used in ostomy procedures. The ostomy pouch 3 can also be customized for specific use with the system of the present invention.

The general configuration of the present invention allows the application of localized compressive force to the stoma mucosa. This inhibits audible vibration frequencies escaping through the stoma, a method also known as Mechanical Vibration Inhibition (MVI). As previously discussed, the ostomy pouch 3 can be any of the currently available pouches in the market that the patient uses. Generally, the ostomy pouch 3 comprises a pouch flange 4, a proximal pouch panel 5, and a distal pouch panel 6, as can be seen in FIGS. 1 through 9. The proximal pouch panel 5 and the distal pouch panel 6 correspond to two thin and flexible panels of equal size that are assembled to form the pouch portion of the ostomy pouch 3. The pouch flange 4 is part of the mechanism that allow the removable attachment of the ostomy pouch 3 to the patient's body. In some embodiments, the pouch flange 4 can be replaced by an integrated skin barrier which also facilitates the attachment of the ostomy pouch 3 to the patient's body while also protecting the skin area surrounding the stoma.

In general, the present invention can be implemented as follows: the proximal pouch panel 5 and the distal pouch panel 6 are hermetically and perimetrically connected to each other, as can be seen in FIGS. 1 through 9. This way, a pouch body is formed with an internal collection space where the discarded waste is collected. In addition, the pouch flange 4 is integrated through the proximal pouch panel 5 to facilitate the removable attachment of the ostomy pouch 3 to the patient's body. The pouch flange 4 delineates an opening on the proximal pouch panel 5 through which the discarded waste falls into the ostomy pouch 3. When a skin barrier is implemented, the opening on the proximal pouch panel is delineated by the skin barrier. Moreover, the first disc-shaped muffler 1 is externally mounted onto the ostomy pouch 3 in such a way that the first disc-shaped muffler 1 coincides with the integral opening on the proximal pouch panel 5 delineated by the pouch flange 4.

As can be seen in FIGS. 1 through 9, the first disc-shaped muffler 1 is positioned concentric to the pouch flange 4 to ensure that the first disc-shaped muffler 1 fully covers the stoma. This allows the first disc-shaped muffler 1 to be aligned with the perimeter of the pouch flange 4 or, alternatively, the skin barrier. Further, the first disc-shaped muffler 1 is externally attached onto the distal pouch panel 6, opposite the proximal pouch panel 5, to secure the first disc-shaped muffler 1 in place within the ostomy pouch 3. Similarly, the second disc-shaped muffler 2 is positioned adjacent to the first disc-shaped muffler 1 so that the second disc-shaped muffler 2 can be stacked on top of the first disc-shaped muffler 1. In addition, the second disc-shaped muffler 2 is also positioned concentric to the pouch flange 4 and the first disc-shaped muffler 1 so that the second disc-shaped muffler 2 fully covers the first disc-shaped muffler 1 and the stoma. Furthermore, the second disc-shaped muffler 2 is attached onto the first disc-shaped muffler 1, opposite to the distal pouch panel 6, to externally attach the second disc-shaped muffler 2 to the first disc-shaped muffler 1 and the ostomy pouch 3. In other embodiments, the system of the present invention can be modified to accommodate other designs of the ostomy pouch 3, such as matching other coupling mechanisms that are different from the pouch flange 4 or the integrated skin barrier.

Different mechanisms can be utilized to facilitate the attachment of the first disc-shaped muffler 1 and the second disc-shaped muffler 2 to the ostomy pouch 3. In the preferred embodiment, a type of adhesive can be used to securely attach each disc-shaped muffler to the ostomy pouch 3. As can be seen in FIGS. 1 through 7, the system of the present invention may further comprise a first quantity of adhesive 7 and a second quantity of adhesive 8. The first quantity of adhesive 7 and the second quantity of adhesive 8 is applied to one of the surfaces of the corresponding disc-shaped muffler to facilitate the attachment of the corresponding disc-shaped muffler to the ostomy pouch 3. Each quantity of adhesive can be applied to the corresponding disc-shaped muffler as a layer of adhesive, as strips of adhesive, etc. Further, a protective layer such as a removable seal can be provided over each quantity of adhesive to protect the corresponding quantity of adhesive until ready for use.

In the preferred embodiment, each quantity of adhesive can be implemented as follows: the first disc-shaped muffler 1 is externally attached onto the distal pouch panel 6 by the first quantity of adhesive 7, as can be seen in FIGS. 1 through 7. The first quantity of adhesive 7 is applied to the surface of the first disc-shaped muffler 1 that is attached to the distal pouch panel 6. Similarly, the second disc-shaped muffler 2 is attached onto the first disc-shaped muffler 1 by the second quantity of adhesive 8. The second quantity of adhesive 8 is also applied to the surface of the second disc-shaped muffler 2 that engages the first disc-shaped muffler 1. In other embodiments, different attachment mechanisms can be utilized to facilitate the attachment of each disc-shaped muffler to the ostomy pouch 3.

As previously discussed, the first disc-shaped muffler 1 and the second disc-shaped muffler 2 are designed to stifle stoma-related sounds to improve the comfort of the patient wearing the ostomy pouch 3. Both the first disc-shaped muffler 1 and the second disc-shaped muffler 2 are preferably made of a flexible foam material. Each foam disc has a specific porosity that gives the corresponding disc-shaped muffler enough flexibility to conform to the stoma. In the preferred embodiment, the first disc-shaped muffler 1 is made from a very malleable and soft foam which contours to the stoma through the distal pouch panel 6 to create a snug but gentle fit against the stoma. The pressure against the stoma prevents the stoma from vibrating when gas is eliminated, which in turn prevents noise from occurring.

The targeted pressure applied by the first disc-shaped muffler 1 is specifically calibrated to achieve MVI while allowing for the normal, unobstructed output of solid and liquid waste into the ostomy pouch 3. Further, the second disc-shaped muffler 2 is also made from soft foam with a higher density that adds a little bit of rigidity to the system. So, the foam density of the first disc-shaped muffler 1 is less than the foam density of the second disc-shaped muffler 2. The greater foam density of the second disc-shaped muffler 2 is necessary to ensure consistent pressure against the stoma is maintained to prevent vibrations and sounds. In other embodiments, different foam densities can be implemented for each disc-shaped muffler.

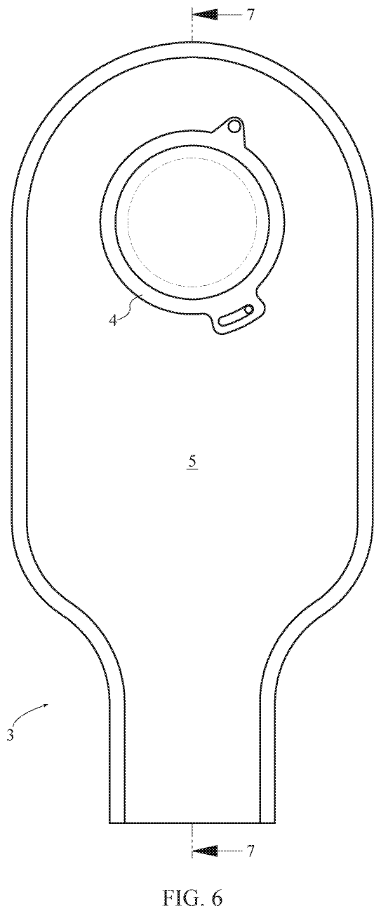

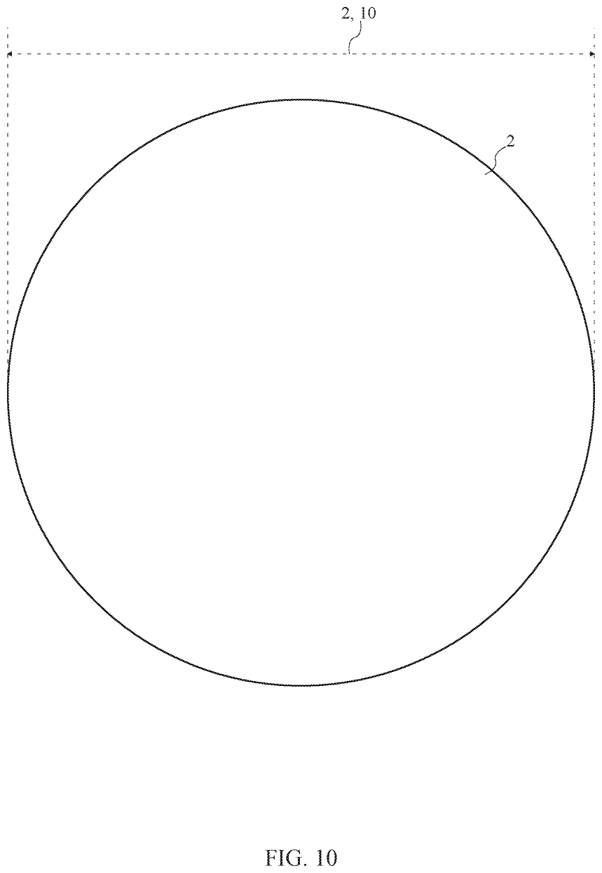

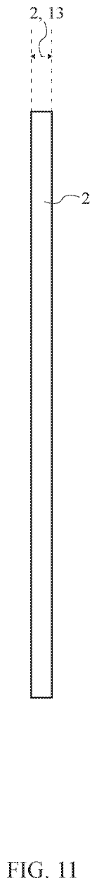

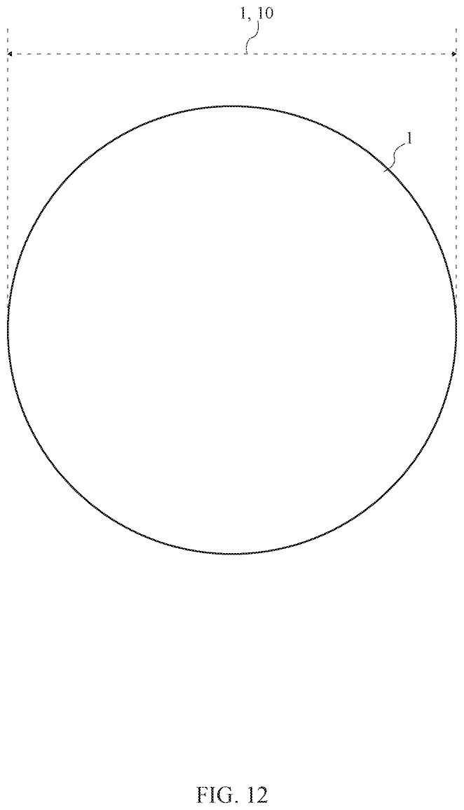

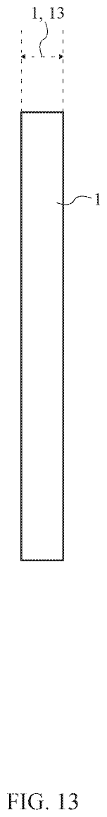

As previously discussed, the first disc-shaped muffler 1 fits within the pouch flange 4 when the first disc-shaped muffler 1 is pressed against the stoma through the distal pouch panel 6. So, a diameter 10 of the first disc-shaped muffler 1 is preferably smaller than an inner diameter 12 of the pouch flange 4 to allow the first disc-shaped muffler 1 to fit within the pouch flange 4 or to sit flush over the stoma opening area, as can be seen in FIGS. 6, 7, and 10 through 13. Further, a diameter 10 of the second disc-shaped muffler 2 is larger than an outer diameter 11 of the pouch flange 4. This way, the second disc-shaped muffler 2 covers the first disc-shaped muffler 1 and the stoma providing support for the first disc-shaped muffler 1 when pressed against the stoma. Alternatively, the diameter 10 of the first disc-shaped muffler 1 can be smaller than the attachment area of a one-piece system and the diameter 10 of the second disc-shaped muffler 2 can be larger than the attachment zone of the ostomy pouch 3. In other embodiments, the overall size and shape of each disc-shaped muffler can be changed to accommodate specific designs of the ostomy pouch 3, the pouch flange 4, and/or the integrated skin barrier.

Further, a ratio between a diameter 10 of the first disc-shaped muffler 1 and a diameter 10 of the second disc-shaped muffler 2 is preferably 10:17, as can be seen in FIGS. 6, 7, and 10 through 13. For example, the diameter 10 of the first disc-shaped muffler 1 can be 50 millimeters (mm), and the diameter 10 of the second disc-shaped muffler 2 can be 85 mm. In addition, a ratio between a thickness 13 of the first disc-shaped muffler 1 and a thickness 13 of the second disc-shaped muffler 2 is preferably 2:1. For example, the thickness 13 of the first disc-shaped muffler 1 can be six mm thick and the thickness 13 of the second disc-shaped muffler 2 can be three mm.

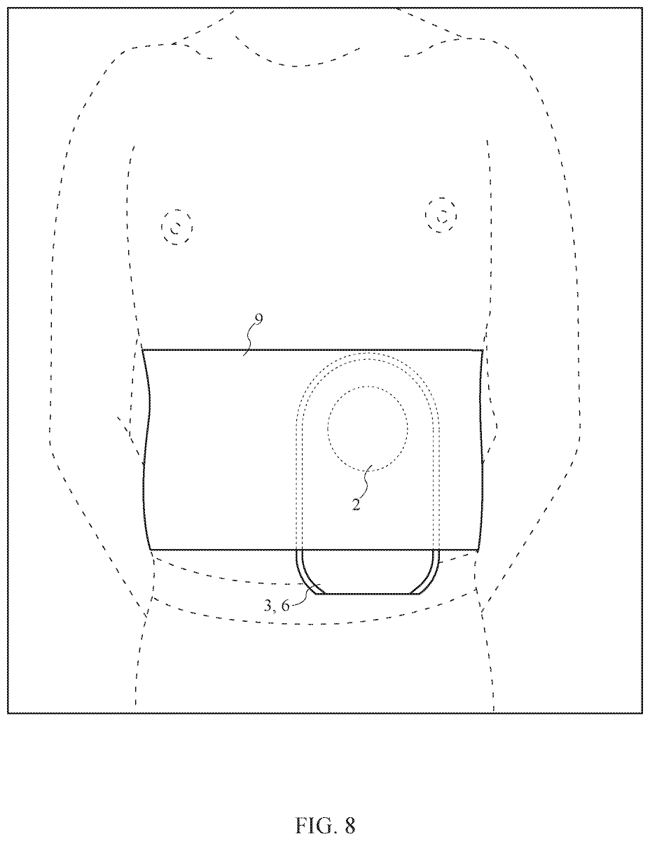

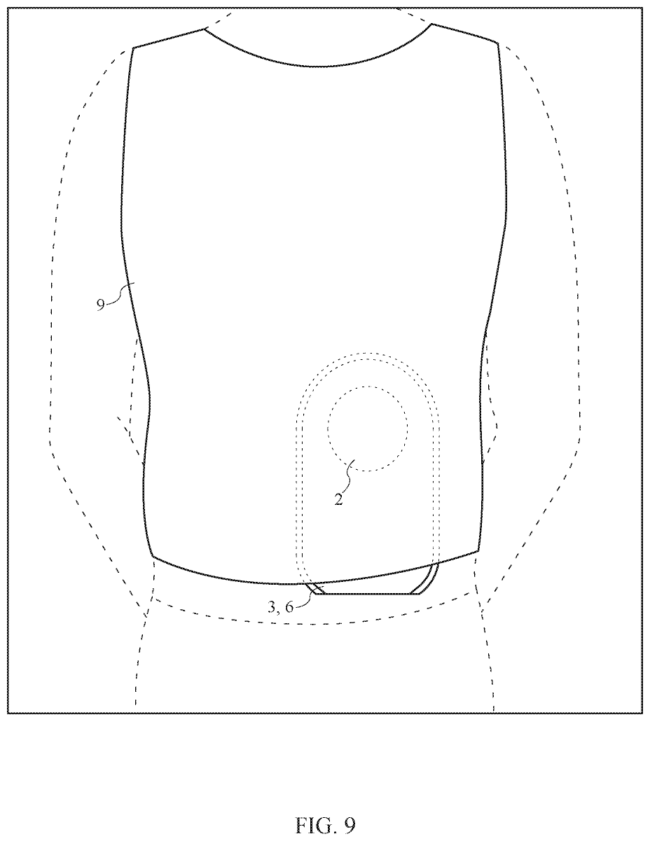

In the preferred embodiment, the system of the present invention may further include means to provide external force to press the first disc-shaped muffler 1 against the stoma through the second disc-shaped muffler 2 and the ostomy pouch 3. In some embodiments, the present invention may further comprise a piece of elastic compression fabric 9 that can be placed over the system to increase the efficiency of stifling stoma-related sounds, as can be seen in FIGS. 8 and 9. For example, the piece of elastic compression fabric 9 can be a compression band that is worn around the patient's body, covering the system, to press the first disc-shaped muffler 1, the ostomy pouch 3, and the second disc-shaped muffler 2 against the stoma. The compression band is preferably an ultra-soft, stretch abdominal bad. Further, the piece of elastic compression fabric 9 can also be a wearable compression garment, such as a compression shirt, that the patient can comfortably wear under other clothes and/or accessories. The wearable compression garment also serves the same purpose as the compression band to press the first disc-shaped muffler 1, the ostomy pouch 3, and the second disc-shaped muffler 2 against the stoma.

The piece of elastic compression fabric 9 can be implemented as follows: the piece of elastic compression fabric 9 is positioned external to the ostomy pouch 3 in such a way that the piece of elastic compression fabric 9 can cover the first disc-shaped muffler 1, the ostomy pouch 3, and the second disc-shaped muffler 2, as can be seen in FIGS. 8 and 9. To do so, the piece of elastic compression fabric 9 can be different types of elastic compression materials. For example, the piece of elastic compression fabric 9 can be made of a polyester, elastane, and/or spandex-based material. Further, the piece of elastic compression fabric 9 is pressed against the second disc-shaped muffler 2 and the distal pouch panel 6 so that the first disc-shaped muffler 1 is pressed against the stoma. The pressing force is generated by the elastic properties of the piece of elastic compression fabric 9. In other embodiments, the piece of elastic compression fabric 9 can be provided as different wearable devices that help press the system against the stoma while also helping the patient hide the system.

The system of the present invention allows the implementation of a method for inhibiting mechanical vibrations of a stoma to reduce stoma-related noises. The overall process of the method of the present invention includes the steps of positioning a flexible first disc-shaped muffler 1 on the exterior of an ostomy pouch 3, with the first disc-shaped muffler 1 being aligned concentric to a pouch flange 4 or integrated skin barrier of the ostomy pouch 3, as can be seen in FIGS. 1 through 9. A second disc-shaped muffler 2 is then positioned external to the ostomy pouch 3 so that the second disc-shaped muffler 2 is stacked over the first disc-shaped muffler 1. Moreover, a continuous compressive force is applied to the second disc-shaped muffler 2, thereby transmitting a localized pressure through the first disc-shaped muffler 1 and the ostomy pouch 3 directly to the stoma mucosa. The localized pressure is calibrated to achieve MVI by preventing involuntary muscle vibrations surrounding the stoma while permitting the unobstructed output of solid and liquid waste into the ostomy pouch.

The continuous compressive force is applied using different mechanisms. In one embodiment, the continuous compressive force is implemented by donning a piece of elastic compression fabric 9 over the second disc-shaped muffler 2, the first disc-shaped muffler 1, and the ostomy pouch 3. Further, the first disc-shaped muffler 1 is a malleable foam that contours to the stoma through the ostomy pouch 3 to create a snug fit against the stoma, and the second disc-shaped muffler 2 is a higher-density foam that provides rigidity to maintain consistent pressure against the stoma. Furthermore, the localized pressure prevents the stoma from vibrating during the elimination of gas, thereby preventing audible vibration frequencies from escaping through the stoma. In other embodiments, the method of the present invention can be altered to implement other subprocess that accommodate specific designs of the ostomy pouch 3.

Although the invention has been explained in relation to its preferred embodiment, it is to be understood that many other possible modifications and variations can be made without departing from the spirit and scope of the invention as hereinafter claimed.

What is claimed is:

1. A muffling system for an ostomy pouch comprising:
a first disc-shaped muffler;
a second disc-shaped muffler;
an ostomy pouch;
the ostomy pouch comprising a pouch flange, a proximal pouch panel, and a distal pouch panel;
the proximal pouch panel and the distal pouch panel being hermetically and perimetrically connected to each other;
the pouch flange being integrated through the proximal pouch panel;
the first disc-shaped muffler being positioned concentric to the pouch flange;
the first disc-shaped muffler being externally attached onto the distal pouch panel, opposite the proximal pouch panel;
the second disc-shaped muffler being positioned concentric to the pouch flange and the first disc-shaped muffler; and
the second disc-shaped muffler being attached onto the first disc-shaped muffler, opposite to the distal pouch panel.

2. The muffling system for an ostomy pouch as claimed in claim 1 comprising:
a first quantity of adhesive; and
the first disc-shaped muffler being externally attached onto the distal pouch panel by the first quantity of adhesive.

3. The muffling system for an ostomy pouch as claimed in claim 1 comprising:
a second quantity of adhesive; and
the second disc-shaped muffler being attached onto the first disc-shaped muffler by the second quantity of adhesive.

4. The muffling system for an ostomy pouch as claimed in claim 1 comprising:
a piece of elastic compression fabric;
the piece of elastic compression fabric being positioned external to the ostomy pouch; and
the piece of elastic compression fabric being pressed against the second disc-shaped muffler, the first disc-shaped muffler, and the distal pouch panel.

5. The muffling system for an ostomy pouch as claimed in claim 4, wherein the piece of elastic compression fabric is a compression band.

6. The muffling system for an ostomy pouch as claimed in claim 4, wherein the piece of elastic compression fabric is a wearable compression garment.

7. The muffling system for an ostomy pouch as claimed in claim 1, wherein a diameter of the first disc-shaped muffler is smaller than an inner diameter of the pouch flange.

8. The muffling system for an ostomy pouch as claimed in claim 1, wherein a diameter of the second disc-shaped muffler is larger than an outer diameter of the pouch flange.

9. The muffling system for an ostomy pouch as claimed in claim 1, wherein a ratio between a diameter of the first disc-shaped muffler and a diameter of the second disc-shaped muffler is 10:17.

10. The muffling system for an ostomy pouch as claimed in claim 1, wherein a ratio between a thickness of the first disc-shaped muffler and a thickness of the second disc-shaped muffler is 2:1.

11. The muffling system for an ostomy pouch as claimed in claim 1, wherein the first disc-shaped muffler and the second disc-shaped muffler are made of a flexible foam material.

12. The muffling system for an ostomy pouch as claimed in claim 11, wherein a foam density of the first disc-shaped muffler is less than a foam density of the second disc-shaped muffler.

13. A muffling system for an ostomy pouch comprising:
a first disc-shaped muffler;
a second disc-shaped muffler;
a piece of elastic compression fabric;
an ostomy pouch;
a first quantity of adhesive;
a second quantity of adhesive;
the ostomy pouch comprising a pouch flange, a proximal pouch panel, and a distal pouch panel;
the proximal pouch panel and the distal pouch panel being hermetically and perimetrically connected to each other;
the pouch flange being integrated through the proximal pouch panel;
the first disc-shaped muffler being positioned concentric to the pouch flange;
the first disc-shaped muffler being externally attached onto the distal pouch panel by the first quantity of adhesive, opposite the proximal pouch panel;
the second disc-shaped muffler being positioned concentric to the pouch flange and the first disc-shaped muffler;
the second disc-shaped muffler being attached onto the first disc-shaped muffler by the second quantity of adhesive, opposite to the distal pouch panel;
the piece of elastic compression fabric being positioned external to the ostomy pouch; and
the piece of elastic compression fabric being pressed against the second disc-shaped muffler, the first disc-shaped muffler, and the distal pouch panel.

14. The muffling system for an ostomy pouch as claimed in claim 13, wherein the piece of elastic compression fabric is a compression band.

15. The muffling system for an ostomy pouch as claimed in claim 13, wherein the piece of elastic compression fabric is a wearable compression garment.

16. The muffling system for an ostomy pouch as claimed in claim 13, wherein a diameter of the first disc-shaped muffler is smaller than an inner diameter of the pouch flange.

17. The muffling system for an ostomy pouch as claimed in claim 13, wherein a diameter of the second disc-shaped muffler is larger than an outer diameter of the pouch flange.

18. The muffling system for an ostomy pouch as claimed in claim 13, wherein a ratio between a diameter of the first disc-shaped muffler and a diameter of the second disc-shaped muffler is 10:17.

19. The muffling system for an ostomy pouch as claimed in claim 13, wherein a ratio between a thickness of the first disc-shaped muffler and a thickness of the second disc-shaped muffler is 2:1.

20. The muffling system for an ostomy pouch as claimed in claim 13, wherein the first disc-shaped muffler and the second disc-shaped muffler are made of a flexible foam material, and wherein a foam density of the first disc-shaped muffler is less than a foam density of the second disc-shaped muffler.
